(19) United States
(12) Patent Application Publication
(10) Pub. No.: US 2013/0210095 A1
(43) Pub. Date: Aug. 15, 2013

(54) NOVEL STRAINS OF MICROALGAE OF THE GENUS BOTRYOCOCCUS AND METHOD FOR THE CULTURE OF SAID MICROALGAE IN MIXOTROPHIC MODE

(71) Applicant: Pierre Calleja, Bordeaux (FR)

(72) Inventor: Pierre Calleja, Bordeaux (FR)

(73) Assignee: FERMENTALG, LIBOURNE (FR)

(21) Appl. No.: 13/878,468

(22) PCT Filed: Oct. 27, 2011
(86) PCT No.: PCT/FR11/52524

§ 371 Date: Apr. 9, 2013

(30) Foreign Application Priority Data
Oct. 28, 2010 (FR) 1058912

(51) Int. Cl. C12P 5/00; C12N 1/12; C12R 1/89; C12P 7/64

(52) U.S. Cl. CPC C12P 5/007; C12P 7/6409; C12N 1/12; C12R 1/89
USPC 435/134; 435/167; 435/257.1

(57) ABSTRACT

Novel strains of microalgae which belong to the Botryococcus genus and which can grow in a mixotrophic mode, and a cultivation method which includes providing light in the form of flashes for the production of lipids and hydrocarbons, in particular in the form of botryococcenes, which are useful in the production of biofuel.

The invention relates to a method for the culture of microalgae of the genus Botryococcus which uses a supply of light that is discontinuous or variable over time, in particular, in the form of flashes, as well as novel strains of microalgae of the genus Botryococcus that are particularly suitable for the production of hydrocarbons in mixotrophic mode.

### Preamble

Unicellular algae are in the form of photosynthetic microorganisms of autotrophic character, i.e. they have the capacity to grow autonomously by photosynthesis.

Most species of unicellular algae encountered in freshwater or the oceans are strictly autotrophic, i.e. they cannot grow other than by photosynthesis. For the latter, the presence of carbon-containing substrates or organic matter in their environment is not favourable to them and even tends to inhibit their growth.

However, a certain number of species of unicellular algae, of very varied families and origins are found to be not strictly autotrophic. Some of them, called heterotrophs, are capable of developing in the complete absence of light, by fermentation, i.e. by using organic matter.

Other species of algae, for which photosynthesis is still indispensable to their development, are capable of using both photosynthesis and organic matter present in their environment. These intermediate species, called mixotrophs, can be cultured both in the presence of light and organic matter.

This particular feature of the so-called “mixotrophic” algae seems to be connected with their metabolism, which allows them to carry on photosynthesis and fermentation simultaneously. The two types of metabolism coexist with an overall positive effect on the growth of the algae [Yang C. et al. (2000) Biochemical Engineering Journal 6:87-102].

At present, the classification of algae is still based largely on morphological criteria and on the nature of the photosynthetic pigments that their cells contain. It therefore gives little indication of the autotrophic, heterotrophic or mixotrophic character of the algae, whereas the latter cover a very great diversity of species and forms [Dubinsky et al. 2010, hydrobiologia, 639:153-171]. Therefore, a strain is considered to be mixotrophic if it can be proved experimentally that it has the ability to grow by photosynthesis in a minimum medium, to which a carbon-containing substrate such as glucose, acetate or glycerol is added. If this supplementation with carbon-containing substrate does not give rise to growth inhibition during the illuminated phase, then the strain can be considered as having a mixotrophic character.

Unicellular algae are currently the subject of numerous industrial projects as certain species are capable of accumulating or secreting large quantities of lipids, in particular of polyunsaturated fatty acids.

Under favourable conditions, the microalgae can thus accumulate up to 80% of their dry weight in fatty acids and therefore offer a credible alternative to the culture of oleaginous land plants, in particular for the production of biofuels [Li, Y et al., 2008 Biotechnol. Prog., 24: 815-820].

It is known, moreover, that certain strains of microalgae of the genus Botryococcus (Chlorophyta, Chlorophyceae, Chlorococcales, Disctyosphaericae) [ITIS, Catalogue of Life, 2010] are capable of producing significant quantities of hydrocarbons, in particular n-alkadienes, trienes, methylated squalenes, triterpenoids, tetraterpenoids and lycopadienes. Moreover, these strains produce particular hydrocarbons with long carbon chains, grouped together under the name of botryococcenes. These hydrocarbons consist predominantly of unbranched isoprenoid triterpenes with the formula CₙH₂ₙ₋₁₀. They can be converted to kerosene or gasoil by cracking and refining. A distinction is drawn between different groups of Botryococcus strains (A, B and L) depending on the profile of the lipids constituting the botryococcenes [Metzger, P et al. (2005) Applied Microbiology and Biotechnology 6(25): 486-496].

To date, Botryococcus braunii is the species, which has been studied the most, due to the quality of its hydrocarbons, and the ease with which it is cultured in autotrophic mode. The culture of this green alga, known to be highly/strongly pigmented, generally takes place under bright light conditions, between 500 and 1500 μmol. m⁻². s⁻¹.

The strain B. braunii var. Showa is known to accumulate up to 30% of its dry weight in botryococcenes. The fatty chains of these botryococcenes comprise between 30 and 37 carbon atoms [U.S. Plant Pat. No. 6,169]. The genome of this strain is currently in the process of being sequenced.

A mutant variety of this strain, Botryococcus braunii var. Ninsei has been described as being able to secrete the botryococcenes into the extracellular matrix [US 2006/0265800]. This secretion has the effect of making the colonies of Botryococcus float in their liquid culture medium, which advantageously makes it possible to harvest the algae charged with botryococcenes at the surface of the culture medium.

The yields of lipids obtained using these algae are however currently insufficient to be able to envisage a cost-effective production of hydrocarbons on an industrial scale. In fact, so that the energy balance of the exploitation of the microalgae of the genus Botryococcus is satisfactory, it would be necessary to reduce the energy supplied to the cultures in the form of light, while increasing the quantity and the quality of the lipids or of the hydrocarbons that can be converted to bio-fuels.

In order to achieve this objective, the applicant selected novel strains of Botryococcus, originating from his personal collections. Among these strains he researched those having the ability to grow, both in mixotrophic mode, and in the presence of discontinuous lighting, in particular, in the form of flashes.

The rapid alternation of illuminated phases and dark phases, generally perceived as stressful for micro-algae such as Botryococcus, have unexpectedly made it possible to isolate novel strains of Botryococcus having a greater ability to evolve under mixotrophic conditions and adapt to light fluctuations. These novel strains of Botryococcus, which are capable of resisting repeated changes in light intensity, are particularly suitable for the production of lipids and hydrocarbons in mixotrophic mode. In particular, they tolerate well a discontinuous light supply, the intensity of which is overall less than that required by cultures in autotrophic mode, or under constant illumination. Moreover, part of the energy consumed by the algae in this system is obtained from a supplementation of the culture medium with carbon-containing substrates such as glycerol or acetate, which can be obtained from by-products originating from various industries.

Without being bound by theory, the inventor hypothesizes that the selection of strains by flashing, makes it possible to isolate strains having a mixed metabolism, i.e. better able to simultaneously carry out photosynthesis and fermentation.

Moreover, according to the inventor, it is when the strains pass from one type of metabolism to the other, with the variations in light intensity, that they tend to store lipid reserves, in particular, in the form of hydrocarbons.

The implementation of the selection method according to the invention described hereafter, applied more particularly to strains of microalgae of the Botryococcus genus, has made it possible to isolate three novel strains of the species Botryococcus braunii and another of the species Botryococcus sudeticus, particularly suitable for the production of lipids and hydrocarbons.

The three strains of Botryococcus braunii are original in that one (827) appears to be both mixotrophic and heterotrophic, which is not the case with the other known mixotrophic strains of Botryococcus braunii, and the other two (828 and 829) appear to be strictly mixotrophic (complete inhibition of growth in heterotrophic mode) in the presence of glycerol, saccharose and lactose in their culture medium. The latter two strains, 828 and 829, seem to have the same characteristics.

As for the Botryococcus sudeticus strain 841, it displays strict mixotrophy in the presence of glucose and acetate in culture medium, which distinguishes it from the other strains and from the strains of the state of the art.

Three of these new strains have been deposited according to the Budapest Treaty of 20 Oct. 2010, at the CCAP (Culture Collection of Algae and Protozoa, Scottish Association for Marine Science, Dunstaffnage Marine Laboratory, Oban, Argyll PA371QA, Scotland, United Kingdom).

The two Botryococcus braunii strains, 827 and 828 received the accession numbers CCAP 807/5 and CCAP 807/6 respectively. As for the Botryococcus sudeticus strain, 841 it received the accession number CCAP 807/4.

These strains are found to be particularly suitable for the production of lipids in mixotrophic culture conditions, in particular when cultured in the presence of a light supply, the intensity of which is variable or discontinuous.

The different aspects and advantages of the invention are detailed below.

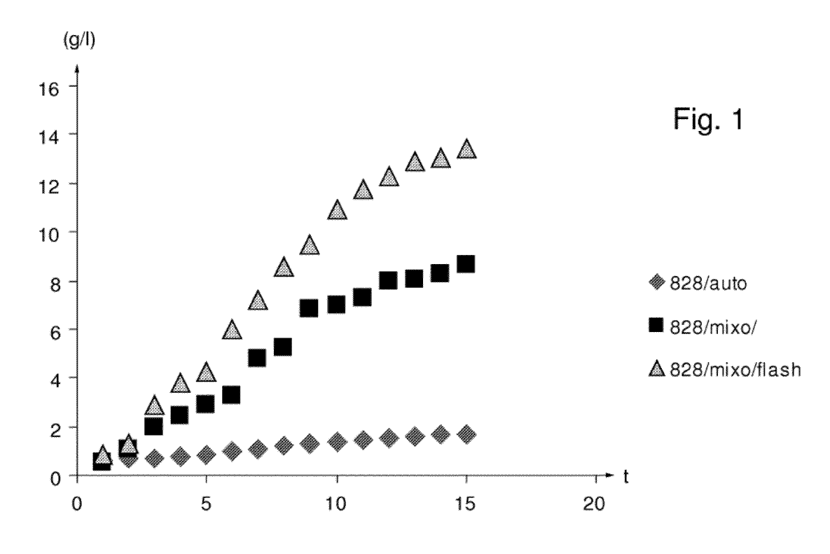

FIG. 1: Graph representing the development of the microalgae biomass (g/L) in culture, in culture medium f/10, supplemented with 10% soil extract over time (days) for the different culture modes: autotrophy, (♦) mixotrophy (▪) and mixotrophy in flash mode according to the invention (Δ). The cultures are produced using the strain 828, as described in Example 2.

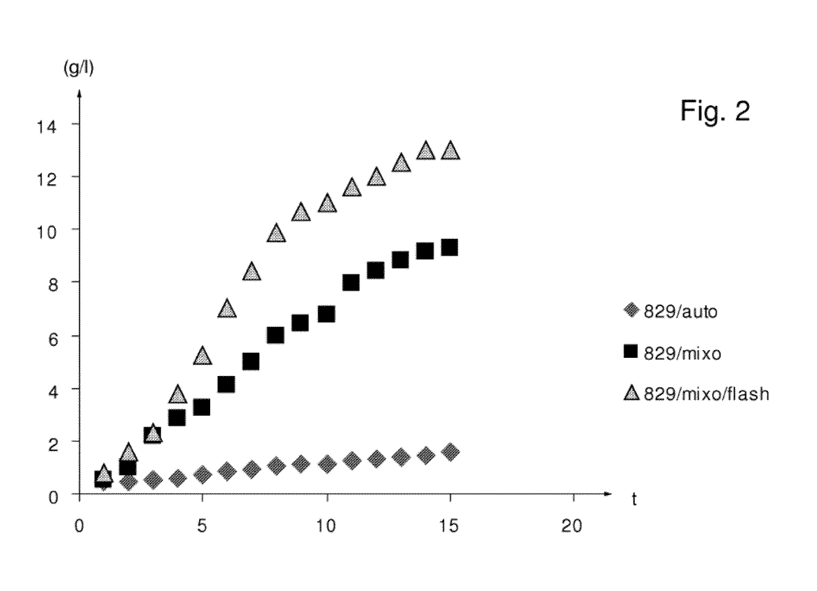

FIG. 2: Graph representing the development of the microalgae biomass (g/L) in culture, in culture medium f/10, supplemented with 10% soil extract over time (days) for the different culture modes: autotrophy, (♦), mixotrophy (▪) and mixotrophy in flash mode according to the invention (Δ). The cultures are produced using the strain 829, as described in Example 2.

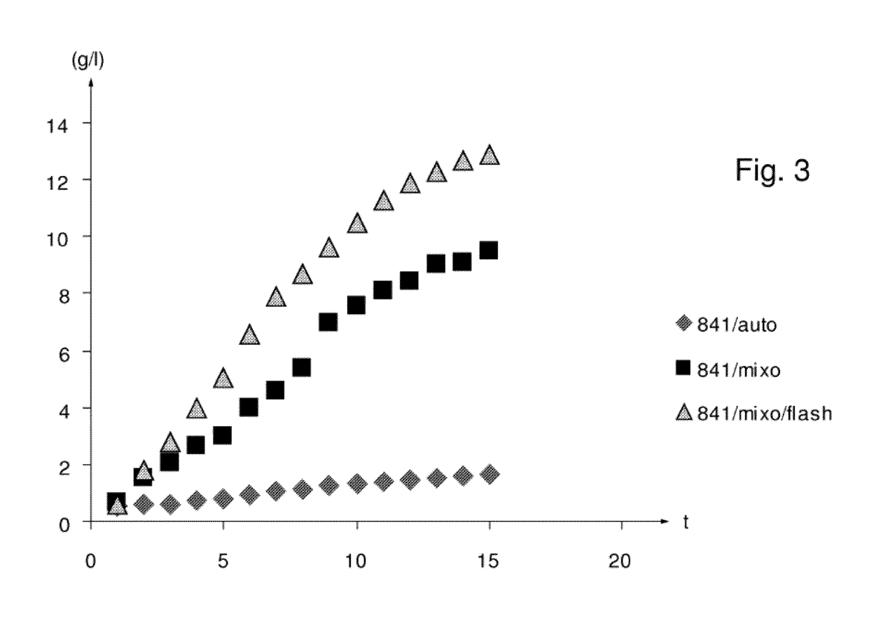

FIG. 3: Graph representing the development of the microalgae biomass (g/L) in culture, in culture medium f/10, supplemented with 10% soil extract over time (days) for the different culture modes: autotrophy (♦), mixotrophy (▪) and mixotrophy in flash mode according to the invention (Δ). The cultures are produced using the strain 841, as described in Example 2.

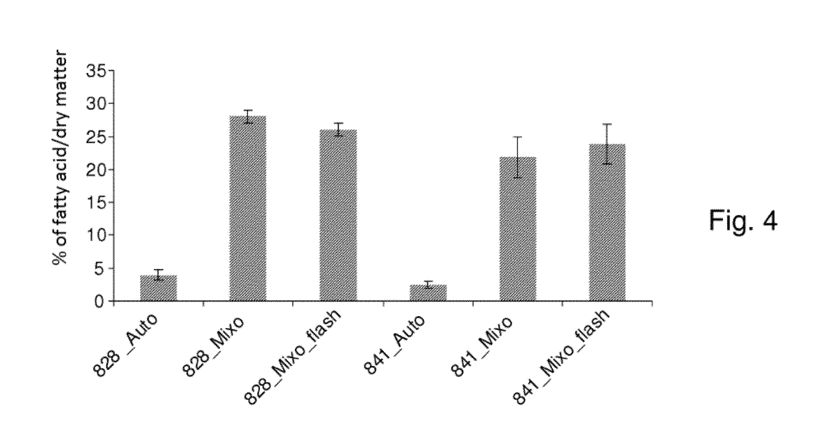

FIG. 4: Diagram representing the fatty acid content of the strains 828 and 841 after 5 days of culture according to the different culture modes (% fatty acids/dry matter) under autotrophic conditions (auto), mixotrophic conditions (mixo) and mixotrophic conditions in flash mode (mixo-flash).

### DETAILED DESCRIPTION

The present invention thus concerns a method making it possible to screen or select strains of unicellular algae (microalgae), in particular of the Botryococcus genus, capable of ensuring a high yield in the production of lipids and hydrocarbons.

The selected algae are capable of growing both in mixotrophic mode, therefore using one or more carbon-containing substrates as an energy source, and taking advantage, by photosynthesis, of a variable or discontinuous supply of light. Algae having these properties are considered as having a higher lipid and hydrocarbon production potential than others.

The invention also concerns a method for the culture of microalgae of the Botryococcus genus, using a variable or discontinuous light supply under conditions similar to those used for the selection of the microalgae.

This method is characterized in that the light flux supplied to the cultured algae is variable or discontinuous over time.

In contrast to conventional wisdom, it was found that variable or discontinuous illumination of the cultures, in particular, in mixotrophic mode, had a favourable effect on the algae development and made it possible, in particular, to increase the production of lipids by the algae.

Without being bound to a theory, the inventor believes that a discontinuous or variable light supply has the effect of causing stress in the algae that is favourable to the synthesis of lipids. In fact, it frequently happens in nature, that algae accumulate lipid reserves so as to withstand environmental stresses.

By discontinuous illumination, it is meant illumination punctuated by periods of darkness. The periods of darkness can occupy more than a quarter of the time, preferably half of the time or more, during which the algae are cultured.

According to a preferred aspect of the invention, the illumination is discontinuous. It supplied, for example, in the form of flashes, i.e. for periods of short duration. The successive phases of illumination are then generally comprised between 5 seconds and 10 minutes, preferably between 10 seconds and 2 minutes, more preferably between 20 seconds and 1 minute.

According to another embodiment of the invention, the illumination can be variable, i.e. the illumination is not interrupted by phases of darkness, but the light intensity varies over time. This light variation can be periodic, cyclic, or even random.

According to the invention, the illumination can vary continuously, i.e. the light intensity is not constant and varies continually over time (dμmol(photons)/dt≠0), in a regulated and controlled manner.

According to the invention, it is also possible to use a light supply combining continuous and discontinuous phases of illumination.

The invention relates, in particular, to a method for culturing unicellular algae, characterized in that said algae are cultured in darkness with a supply of light that is discontinuous or variable over time, the intensity of which, in micromoles of photons, varies with an amplitude equal to or greater than 10 μmol. m⁻². s⁻¹, at a rate of several times per hour, preferably equal to or greater than 40 μmol. m⁻². s⁻¹, more preferably, equal to or greater than 50 μmol. m⁻². s⁻¹. The common feature of these different modes of illumination, discontinuous or variable, resides in the fact that, according to the invention, the light intensity supplied to the algae in culture, expressed in micromoles of photons per second per square metre (μmol. m⁻². s⁻¹), varies at least once within the same hour. The amplitude of this variation in light intensity is generally greater than 10 μmol. m⁻². s⁻¹, preferably, greater than or equal to 20 μmol. m⁻². s⁻¹, more preferably, greater than or equal to 50 μmol. m⁻². s⁻¹. In other words, each hour, preferably several times an hour, the light intensity reaches a high and a low value, the difference between which is equal to or greater than that indicated above. Preferably, said light intensity successively reaches the values 50 μmol. m⁻². s⁻¹ and 100 μmol. m⁻². s⁻¹ each hour, more preferably, the values 0 and 50 μmol. m². s⁻¹, even more preferably, the values 0 and 100 μmol. m⁻². s⁻¹.

Note that 1 μmol. m⁻². s⁻¹ corresponds to 1 μE m⁻². s⁻¹ (Einstein), a unit used in the examples in the present application.

The supply of light to the cultures can be provided by lamps distributed around the external wall of the fermenters. A clock switches on these lamps for defined illumination times. The fermenters are preferably located in a chamber shielded from daylight, the ambient temperature of which can be controlled.

The method of selection and culture according to the invention applies more particularly to the microalgae of the genus Botryococcus in order to select strains with a high lipid yield.

The method of culture is characterized in that it comprises one or more of the following steps:
the culture of different strains of the genus Botryococcus in the darkness with a supply of light that is discontinuous or variable over time, the intensity of which in micromoles of photons preferably varies in amplitude equal to or greater than 50 μmol. m⁻². s⁻¹, at a rate of at least once per hour;
the maintenance of said culture over several generations;
the isolation of the strain or strains, the number of cells of which has increased the most in the course of said generations.

The aim of the culture method according to the invention is to increase the production of lipids and/or hydrocarbons, in particular, via the recovery of the lipids and hydrocarbons contained in or excreted by the microalgae, more particularly, the botryococcene-type hydrocarbons.

In order to carry out the screening of strains, different strains of microalgae, in particular of the Botryococcus genus, can be cultured, in parallel, on microplates in the same chamber with precise monitoring of the conditions and the development of the different cultures. It is, thus, easy to know the response of the different strains to the discontinuous illumination and, if appropriate, to the addition of one or more carbon-containing substrates to the culture medium. The strains which respond favourably to the discontinuous illumination and to the carbon-containing substrates, generally offer a better yield for the production of lipids and hydrocarbons in terms of quality (lipid profile) and quantity (total lipids or hydrocarbons produced).

Alternatively, the microalgae can be selected in a fermenter from a pool of diverse microalgae, and from which it is sought to select the variants given advantage by the mode of selection according to the invention, combining discontinuous or variable light with mixotrophic culture conditions. In this case, the culture is carried out keeping the microalgae in culture for a number of generations, followed by isolating the constituents that have become the majority in the culture medium throughout the culture period.

The culture method according to the invention is characterized more particularly, in that the culture of the strains is carried out over several generations, preferably in mixotrophic mode, and, in that the cells charged with lipids or hydrocarbons are harvested.

Within the meaning of the present invention, a species of alga is regarded as being mixotrophic provided it can be cultured in the light, in a minimum medium (for example MM or f/10 supplemented with 10% soil extract) to which a carbon-containing substrate is added, for example, with carbon or glycerol concentration equivalent to or greater than 5 mM, without observing any inhibition of growth, i.e. without finding a loss of biomass in dry weight, relative to a culture carried out in the identical minimum medium, lacking a carbon-containing substrate (i.e. in autotrophic mode).

The preferred carbon-containing substrates include acetate, glucose, cellulose, starch, lactose, saccharose and glycerol. The products originating from the bioconversion of starch, for example from corn, wheat or potato, in particular, the starch hydrolysates which are constituted by small molecules, are carbon-containing substrates of choice.

Preferably, the microalgae are chosen from the species Botryococcus braunii and Botryococcus sudeticus. To which species the microalgae belongs is established on the basis of the usual criteria for the classification of microalgae.

The invention also relates to strains of microalgae with a high yield of hydrocarbons and/or lipids, susceptible of being selected according to the method of the invention, characterized in that they are mixotrophic and can grow in discontinuous or variable light.

As indicated by the examples of the present application, the implementation of the method according to the invention has, more particularly, made it possible to isolate novel strains of the Botryococcus genus. These strains, which were deposited in the CCAP collection (Culture Collection of Algae and Protozoa), on 20 Oct. 2010, according to the Budapest Treaty, are as follows:
Botryococcus braunii strain 827, deposited under number CCAP 807/5.
Botryococcus braunii strain 828, deposited under number CCAP 807/6.

These two strains have the characteristic of being mixotrophic but are not heterotrophic, i.e. they can be cultured in minimum medium supplemented with carbon-containing substrate, in the presence of a light supply, but not in the absence of light. This behaviour is observed, in particular, when the carbon-containing substrate added to the culture medium is saccharose, lactose or glycerol. To the inventor's knowledge, this is the first time that strains of Botryococcus exhibit this characteristic.
Botryococcus sudeticus strain 841, deposited under number CCAP 807/4.

This strain of the species sudeticus has the characteristic of being mixotrophic. To the applicant's knowledge, this is the first strain of this species described as being mixotrophic. Moreover, as is the case for the previous strains, this strain has the characteristic of being mixotrophic without being heterotrophic. This is observed, in particular, when the carbon-containing substrate added to the minimum culture medium is glucose or acetate.

Thus, as the applicant has found, the fact that the strains thus selected have good growth capabilities in mixotrophic mode, in the presence of a discontinuous light, predisposes these strains to higher lipid and hydrocarbon production, in particular botryococcene production.

Nevertheless, the culture method according to the invention is applicable to any strain of the genus Botryococcus that can be cultured under mixotrophic conditions, and is not limited solely to the use of the novel strains described in the present application. In fact, the inventors have been able to observe a gain in productivity in the cultures, in particular, in terms of biomass, in all the strains of Botryococcus previously identified as being able to grow under mixotrophic conditions, compared with the same cultures produced in autotrophic mode.

The purpose of the following examples is to complete the description and illustrate the invention. They do not limit the invention in any way.

### Example 1

### 1—Strains

The Botryococcus strains were selected from a collection of strains of the applicant constituted by strains taken from freshwater, isolated and characterized according to common criteria [Komarek, J. et al. (1992) P. Morphological differences in natural populations of the genus Botryococcus (chlorophyceae). Archiv für Protistenkunde, 141(1-2):65-100] [Dayananda C. et al. (2007) Isolation and characterization of hydrocarbon producing green alga Botryococcus braunii from Indian freshwater bodies. Elect. J. Biotechnol., 10:1-14].

### 2—Culture Conditions

Several isolates of Botryococcus braunii and of Botryococcus sudeticus were firstly cultured at 22° C. under autotrophic conditions (200 μE of light) in liquid Minimum Medium (MM) [50 mL/L of Beijerink Solution (NH₄Cl 8 g/L, CaCl₂ 1 g/L, MgSO₄ 2 g/L), 1 mL/L of Phosphate Buffer (K₂HPO₄ 106 g/L KH₂PO₄ 53 g/L), 1 mL/L of a solution of trace elements (BO₃H₃ 11.4 g/L, ZnSO₄ 7H₂O 22 g/L, MnCl₂ 4H₂O 5.06 g/L, FeSO₄ 7H₂O 4.99 g/L, CoCl₂ 6H₂O 1.61 g/L, CuSO₄ 5H₂O 1.57 g/L, Mo₇O₂₄(NH₄)₆ 4H₂O 1.1 g/L, EDTA 50 g/L), 2.42 g/l of Trizma base, pH adjusted between 7.2 and 7.4 with HCl, 1.2 m g/L of Vitamin B₁ and 0.01 mg/L of Vitamin B₁₂ (added extemporaneously)].

Cultures were carried out in mixotrophic mode (200 μE) in continuous and discontinuous light, as well as in heterotrophic mode (control at 0 μE of light) at 22° C. on medium MM with the addition of carbon-containing substrates: acetate 1 g/L, glucose 5 g/L, lactose 10 g/L, saccharose 10 g/L or glycerol 5 g/L.

The heterotrophic and/or mixotrophic character of the Botryococcus strains was evaluated by culturing the microalgae strains in medium MM+ carbon-containing substrate in 24-well microplates (V=2 mL). Growth under autotrophic conditions (MM) was systematically monitored to serve as reference for cultures under mixotrophic and heterotrophic conditions.

The 24-well microplates were placed in an incubation chamber (SANYO MLR-351H) at 22° C., 60% humidity and 200 μE light intensity, in the case of the cultures under autotrophic and mixotrophic conditions, and in an incubation chamber (BINDER KB53) at 22° C., 60% humidity and in darkness (0 μE), in the case of the cultures under heterotrophic conditions.

The cell growth was evaluated by comparison of the turbidity and/or the chlorophyll content with respect to the reference under autotrophic conditions. Monitoring was carried out twice-weekly for a period of 2 weeks in the case of the cultures under autotrophic and mixotrophic conditions, and for a duration of 2 to 3 weeks, in the case of the cultures under heterotrophic conditions. The mobility and the pigmentation of the microalgae cultured under autotrophic/mixotrophic conditions (auto/mixo) and heterotrophic conditions were observed and compared using a binocular microscope, 10× and 32× objectives.

For the cultures carried out in mixotrophic mode in discontinuous light, the light supply consisted of flashes at a rate of 30 flashes of 30 seconds per hour.

The strains for which culture in discontinuous light proved more favourable than in continuous light, were selected. Among these strains, 4 were more particularly studied: 3 strains of Botryococcus braunii (827, 828 and 829) and a strain of Botryococcus sudeticus (841).

### 3—Properties of the Four Selected Strains of the Genus Botryococcus

The effect of light (autotrophy and mixotrophy columns) and of carbon-containing substrates (mixotrophy and heterotrophy columns) such as glucose (Glc 5 g/L), acetate (Ac 1 g/L), saccharose (Sac 10 g/L), lactose (Lac 10 g/L) and glycerol (Gly 5 g/L) on the growth of 4 strains of the genus Botryococcus was evaluated by screening in 24-well microplates on liquid medium MM (cf. table below). The growth is monitored twice weekly for 3 to 4 weeks, by macroscopic observation of the cultures and observation with a binocular microscopic (10× and 32× objectives).

TABLE 1
growth of the selected strains under mixotrophic conditions

| | | | Mixotrophy | | | | | Heterotrophy | | | | |
|---|---|---|---|---|---|---|---|---|---|---|---|---|
| Strains | | | Glc | Ac | Sac | Lac | Gly | Glc | Ac | Sac | Lac | Gly |
| Species | No. | Auto | 5 g/L | 1 g/L | 10 g/L | 10 g/L | 5 g/L | 5 g/L | 1 g/L | 10 g/L | 10 g/L | 5 g/L |
| B. braunii | 827 | + | + | + | + | + | + | + | + | +++ | ++ | + |
| B. braunii | 828 | + | + | + | +++ | + | + | + | + | − | − | − |
| B. braunii | 829 | + | + | + | +++ | + | + | ++ | + | − | − | − |
| B. sudeticus | 841 | + | + | +++ | +++ | + | + | − | − | +++ | + | + |

−: inhibited growth;
+: moderate growth;
++: significant growth;
+++: strong growth

Of the 4 strains of the genus Botryococcus tested, the 2 strains B. braunii 827 and B. sudeticus 841 display a strict heterotrophic character in the presence of saccharose. The other 2 strains, B. braunii 828 and B. braunii 829 are strictly mixotrophic at 200 μE in the presence of saccharose, and the strain B. sudeticus 841 is strictly mixotrophic at 200 μE in the presence of acetate. It is, in fact, observed that the 2 strains B. braunii 827 and B. sudeticus 841 have significant growth at 0 μE in the presence of 10 g/L of saccharose, growth greater than that under autotrophic conditions. Increased growth is also observed at a light intensity of 200 μE in the case of the strains B. braunii 828 and B. braunii 829, when 10 g/L of saccharose is added to the culture medium, and increased growth of the strain B. sudeticus 841 when 1 g/L of acetate is added to the culture medium, compared to their growth under autotrophic conditions (light intensity of 200 μE).

### 4—Conclusion

This study has made it possible to demonstrate novel strains of Botryococcus braunii and Botryococcus sudeticus having a mixotrophic character vis-à-vis certain carbon-containing substrates. The addition of carbon-containing substrates such as saccharose and acetate significantly improves the respective growth of these strains.

### Example 2

### 1—Culture of the Strains of Botryococcus in a Bioreactor According to the Mixo/Flash Method

The cultures of each of the strains isolated in Example 1 (828, 829 and 841) were carried out in 2-litre fermenters (bioreactors) used with dedicated automatic equipment and with computerized supervision. The pH of the system was adjusted by adding base (solution of sodium hydroxide at 1N) and/or acid (solution of sulphuric acid at 1N). The culture temperature was fixed at 23° C. Stirring was provided by 3 stirring rotors, mounted on the shaft according to the Rushton configuration (three-blade propellers with down pumping). The stirring speed and the aeration flow rate were regulated to a minimum of 100 rpm and a maximum of 250 rpm with Qₘᵢₙ=0.5 vvm/Q_{max.}=2 vvm respectively. The bioreactor is equipped with an external lighting system surrounding the transparent tank. The intensity, as well as the light cycles, are controlled and regulated by dedicated automatic equipment and with computerized supervision.

The supply of the light to the cultures in the bioreactor was obtained from LED lamps distributed around the outer wall of the fermenters. A clock switches on these LED lamps for illumination times or pulses between 8 and 50 μE. The light intensity of the flash system used under mixotrophic conditions is equal to that used under autotrophic conditions (control).

The reactors were inoculated with a preculture carried out on a mixing table (140 rpm) in a controlled temperature chamber (22° C.), illuminated continuously at 100 μE. Pre-cultures and cultures were carried out in bioreactors in f/10 medium supplemented with 10% oil extract and 10 mM NaHCO₃. The carbon-containing substrate used for the culture under mixotrophic conditions in the bioreactor is sodium acetate at concentrations comprised between 20 mM and 50 mM.

### 2—Monitoring the Cultures

The concentration of total biomass was monitored by measuring the dry mass (filtration on a GFC filter, Whatman, then drying in an oven under vacuum, 65° C. and −0.8 bar, for at least 24 h before weighing).

The quantification of the total lipids was carried out on cell samples (10⁷ cells/mL) extracted from 5-day-old cultures. The lipids were extracted according to the lipid extraction methods described by Bligh, E. G. and Dyer, W. J. [A rapid method of total lipid extraction and purification (1959) Can. J. Biochem. Physiol 37:911-917].

### 3—Results

### Biomass Development:

The biomass measurements carried out in the different samples, taken daily from the cultures for 15 days, are presented in the graphs in FIGS. 1 to 3 for each of the strains 828, 829 and 841. The graphs allow one to compare the development of the dry biomass per volume of culture in the different culture modes: autotrophic, mixotrophic (continuous light) and mixotrophic with flash.

These measurements show that, for each of the strains, a substantial gain in biomass is obtained in flash mode. The increase is of the order of 30%, relative to mixotrophy in continuous light, and of the order of 120%, relative to autotrophic mode.

### Lipid Content:

The ratio of the quantity of fatty acids present in the cells after 5 days of culture, to the total dry matter, was established for each culture mode: autotrophy (auto), mixotrophy (mixo) and mixotrophy in flash mode (mixo-flash).

The results are represented in the form of diagrams in FIG. 4, for the two strains analyzed, 828 and 841.

These results show that, when the microalgae are cultured in mixotrophic mode, the fatty acid content of the strains increases by a factor of 4 to 5, relative to the cultures in autotrophic mode. The content obtained in mixotrophic mode with flash and without flash is comparable after 5 days.

1. Method for the culture of microalgae of the genus Botryococcus for the production of lipids or hydrocarbons, characterized in that it comprises the following steps:
the culture of one or more strains of microalgae of the genus Botryococcus in darkness in the presence of a supply of light that is discontinuous or variable over time, the intensity of which, in micromoles of photons, varies by an amplitude of more than 10 μmol. m⁻². s⁻¹, at a rate of at least once per hour;
the maintenance of said culture over several generations in the presence of a carbon-containing substrate in the culture medium;
the harvesting of the cells charged with hydrocarbons or lipids.

2. Method according to claim 1, characterized in that the supply of light is discontinuous.

3. Method according to claim 1, characterized in that the supply of light varies by more than 40, preferably, more than 50 μmol. m⁻². s⁻¹.

4. Method according to claim 1, characterized in that it also comprises the recovery of the lipids or hydrocarbons contained in or excreted by the microalgae.

5. Method according to claim 4, characterized in that the hydrocarbons contained in or excreted by the microalgae comprise botryococcenes.

6. Method according to claim 1, characterized in that the culture medium is a minimum medium comprising a carbon-containing substrate.

7. Method according to claim 1, characterized in that the carbon-containing substrate comprises acetate, glucose, cellulose, starch, lactose, saccharose or glycerol.

8. Method according to claim 1, characterized in that the supply of light is carried out in the form of flashes.

9. Method according to claim 8, characterized in that said flashing consists of successive illumination phases of a duration comprised between 5 seconds and 10 minutes, preferably, between 10 seconds and 2 minutes, more preferably, between 20 seconds and 1 minute.

10. Method according to claim 1, characterized in that the microalgae are chosen from the species Botryococcus braunii.

11. Method according to claim 1, characterized in that the microalgae are chosen from the species Botryococcus sudeticus.

12. Microalgae which can be cultured according to the method of claim 1, corresponding to one of the following strains deposited at the CCAP:
Botryococcus sudeticus strain deposited under No. CCAP 807/4;
Botryococcus braunii strain deposited under No. CCAP 807/5; or
Botryococcus braunii strain deposited under No. CCAP 807/6.

13. Method according to claim 2, characterized in that it also comprises the recovery of the lipids or hydrocarbons contained in or excreted by the microalgae.
